(19) United States
(12) Patent Application Publication
(10) Pub. No.: US 2025/0022592 A1
(43) Pub. Date: Jan. 16, 2025

(54) INFORMATION PROCESSING DEVICE, INFORMATION PROCESSING METHOD, AND NON-TRANSITORY COMPUTER READABLE STORAGE MEDIUM

(71) Applicant: NIHON KOHDEN CORPORATION, Tokyo (JP)

(72) Inventor: Kou ADACHI, Tokorozawa-shi (JP)

(21) Appl. No.: 18/769,661

(22) Filed: Jul. 11, 2024

(30) Foreign Application Priority Data
Jul. 12, 2023 (JP) 2023-114472

(51) Int. Cl. G16H 40/63; G06F 3/14

(52) U.S. Cl. CPC G16H 40/63; G06F 3/14

(57) ABSTRACT

An information processing device includes an input interface to which physiological information on a subject is input, at least one processor, and a memory configured to store at least one command. In a case where the at least one command is executed by the processor, the information processing device is configured to classifies the physiological information input to the input interface into a first physiological information group and a second physiological information group, and generates display data including the first physiological information group, the second physiological information group, a first line that is a timeline, and a second line that indicates a value related to the physiological information, and the display data is generated in a form in which the first physiological information group and the second physiological information group are displayed in a state where the first lines overlap each other and the second line is common.

### CROSS-REFERENCE TO RELATED APPLICATIONS

This application is based on and claims priority under 35 USC 119 from Japanese Patent Application No. 2023-114472 filed on Jul. 12, 2023, the entire content of which is incorporated herein by reference.

### TECHNICAL FIELD

The present disclosure relates to an information processing device, an information processing method, and a non-transitory computer readable storage medium.

### BACKGROUND ART

JP2019-150560A discloses a device that displays physiological information on a subject that changes over time and an arrow indicating an increase or decrease in a value of the physiological information.

In the medical field, it is required to quickly notice a condition change of a subject and perform an appropriate treatment before the condition of the subject deteriorates. On the other hand, the condition of the subject often changes rapidly, but even in such a case, the condition of the subject often changes gradually before the condition of the subject changes rapidly. For this reason, it is preferable that it is possible to recognize early that the condition of the subject changes gradually.

### SUMMARY OF INVENTION

Aspect of non-limiting embodiments of the present disclosure relates to provide an information processing device, an information processing method, and a non-transitory computer readable storage medium capable of supporting a medical worker to quickly notice a condition change of a subject.

Aspects of certain non-limiting embodiments of the present disclosure address the features discussed above and/or other features not described above. However, aspects of the non-limiting embodiments are not required to address the above features, and aspects of the non-limiting embodiments of the present disclosure may not address features described above.

According to a first aspect of the present disclosure, there is provided an information processing device including:
an input interface to which physiological information on a subject is input;
at least one processor; and
a memory configured to store at least one command that the processor is configured to execute,
in which in a case where the at least one command is executed by the processor, the information processing device is configured to:
   classifies the physiological information input to the input interface into a first physiological information group and a second physiological information group, the first physiological information group including the physiological information during at least a first period, the second physiological information group including the physiological information during a second period different from the first period; and
   generates display data including the first physiological information group, the second physiological information group, a first line that is a timeline, and a second line that indicates a value related to the physiological information; and
the display data is generated in a form in which the first physiological information group and the second physiological information group are displayed in a state where the first lines overlap each other and the second line is common.

According to a second aspect of the present disclosure, there is provided an information processing method executed by an information processing device including:
classifying physiological information of a subject input to an input interface of the information processing device into a first physiological information group and a second physiological information group, the first physiological information group including the physiological information during at least a first period, the second physiological information group including the physiological information during a second period different from the first period; and
generating display data including the first physiological information group, the second physiological information group, a first line that is a timeline, and a second line that indicates a magnitude of a value of the physiological information,
in which the display data is generated in a form in which the first physiological information group and the second physiological information group are displayed in a state where the first lines overlap each other and the second line is common.

According to a third aspect of the present disclosure, there is provided a non-transitory computer readable storage medium storing a computer program including at least one command which, when executed by a processor of an information processing device, causes the information processing device to:
classify physiological information of a subject input to an input interface of the information processing device into a first physiological information group and a second physiological information group, the first physiological information group including the physiological information during at least a first period, the second physiological information group including the physiological information during a second period different from the first period; and
generate display data including the first physiological information group, the second physiological information group, a first line that is a timeline, and a second line that indicates a magnitude of a value of the physiological information,
in which the display data is generated in a form in which the first physiological information group and the second physiological information group are displayed in a state where the first lines overlap each other and the second line is common.

### BRIEF DESCRIPTION OF DRAWINGS

Exemplary embodiment(s) of the present invention will be described in detail based on the following figures, wherein:

FIG. 1 is a schematic diagram of an information processing system according to an embodiment.

FIG. 2 is a flowchart illustrating an information processing method related to generation of display data.

FIG. 3 is a diagram illustrating an image based on the display data.

FIG. 4 is a flowchart illustrating an information processing method related to generation of alert information and sudden change information.

FIG. 5 is a flowchart illustrating an information processing method related to generation of display data.

FIG. 6 is a diagram illustrating an image based on the display data.

FIG. 7 is a flowchart illustrating an information processing method related to generation of display data.

FIG. 8 is a diagram illustrating an image based on the display data.

FIG. 9 is a flowchart illustrating an information processing method related to generation of display data.

FIG. 10 is a diagram illustrating an image based on the display data.

### DESCRIPTION OF EMBODIMENTS

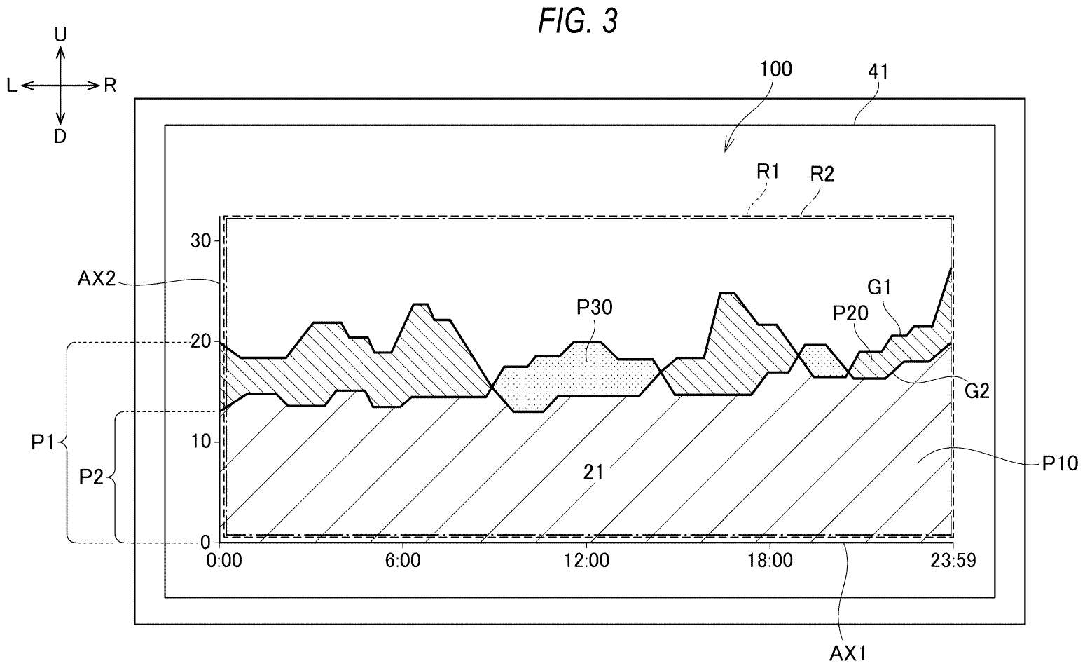

Hereinafter, examples of embodiments of the present disclosure will be described with reference to the drawings. In the description of the present embodiment, a “left-right direction” and an “up-down direction” may be appropriately referred to for convenience of description. These directions are relative directions set for a display unit 41 of a terminal device 40 illustrated in FIGS. 3, 6, 8, and 10. Here, the “left-right direction” is a direction including a “left direction” and a “right direction”. The “up-down direction” is a direction including an “up direction” and a “down direction”. A reference character U illustrated in each drawing indicates the up direction. A reference character D indicates the down direction. A reference character L indicates the left direction. A reference character R indicates the right direction.

### First Embodiment

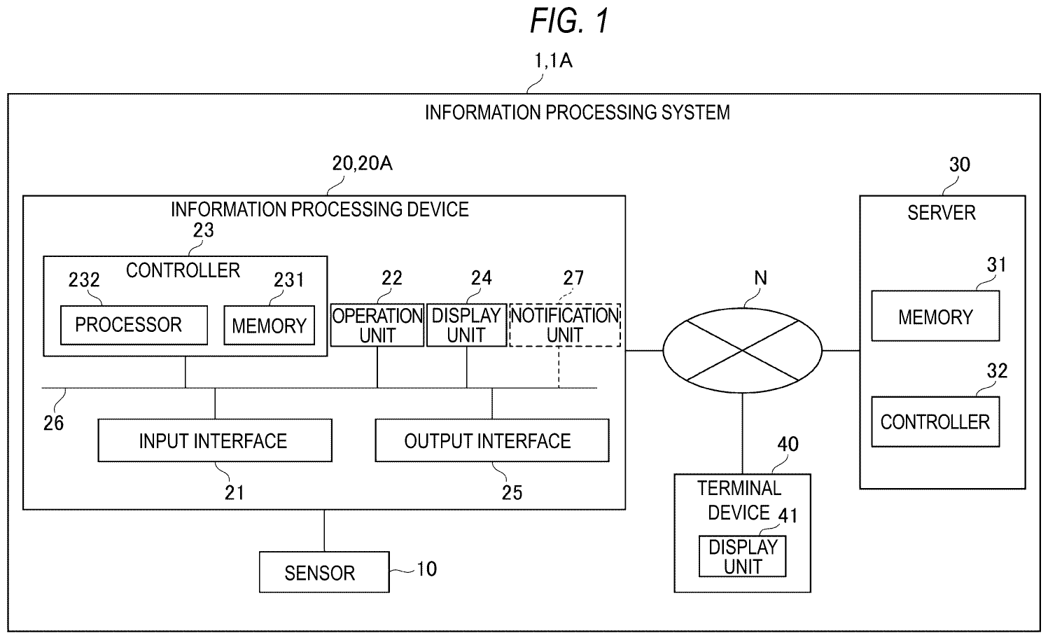

An information processing system 1 according to the present embodiment will be described with reference to FIG. 1. FIG. 1 is a schematic diagram illustrating the information processing system 1 according to the present embodiment. The information processing system 1 is used in, for example, a medical facility such as a hospital. As illustrated in FIG. 1, the information processing system 1 can include a sensor 10, an information processing device 20, a server 30, and the terminal device 40. The sensor 10 and the information processing device 20 are connected by wire or wirelessly. The information processing device 20, the server 30, and the terminal device 40 are communicably connected via a network N. The network N is, for example, a computer network such as a cloud network. The cloud network is a computer network existing in an infrastructure of cloud computing.

The sensor 10 is configured to measure physiological information on the subject. The sensor 10 includes, for example, a blood pressure sensor, a respiration sensor, an SpO2 sensor, a body temperature sensor, and a heart rate sensor. The sensor 10 may further include another sensor different from these sensors. The blood pressure sensor is configured to measure blood pressure (systolic blood pressure, diastolic blood pressure, and mean blood pressure) of the subject. The blood pressure sensor is, for example, a blood pressure monitor or a catheter for measuring a vital blood pressure value. The respiration sensor is configured to measure the respiration rate or the like of the subject. The respiration sensor is, for example, an electrode for measuring an impedance respiration rate. The SpO2 sensor is configured to measure the transcutaneous oxygen saturation (SpO2) of the subject. The SpO2 sensor is, for example, a probe for measuring SpO2. The body temperature sensor is configured to measure the body temperature of the subject. The body temperature sensor is a body temperature meter for measuring the body temperature of the subject, a body temperature probe connected to the body temperature meter, or the like. The heart rate sensor is configured to measure the heart rate of the subject. The heart rate sensor is, for example, an electrode for measuring a heart rate. In the present embodiment, the sensor 10 is configured to measure the physiological information by a passive method, but may be configured to measure the physiological information by an active method. In the measurement of physiological information by the passive method, for example, physiological information is measured by receiving a signal from an object without transmitting a signal such as light or electromagnetic waves from the sensor 10 to the object. In the measurement of physiological information by the active method, for example, a signal is transmitted from the sensor 10 to an object, and the physiological information is measured by receiving a signal reflected by the object or transmitted through the object. The physiological information measured by the sensor 10 is input to the information processing device 20 as an electric signal.

The information processing device 20 is, for example, a bed-side monitor. The information processing device 20 can include an input interface 21, an operation unit 22, a controller 23, a display unit 24, and an output interface 25. These components are communicably connected to each other via a bus 26.

The input interface 21 is configured to receive the physiological information on the subject. In the present embodiment, physiological information measured by the sensor 10 is input to the input interface 21. The input interface 21 is configured to transmit the input physiological information to the controller 23 and the server 30.

The operation unit 22 is configured to receive an input operation by a user. The user is, for example, a medical worker such as a doctor or a nurse. The operation unit 22 is, for example, a touch panel disposed on the display unit 24 in an overlapping manner, an operation button attached to a case, or the like. For example, in a case where the user performs an input operation, on the operation unit 22, for obtaining desired information from the server 30, the operation unit 22 generates a request signal for obtaining desired information from the server 30, based on the input operation. The operation unit 22 is configured to transmit the generated request signal to the server 30.

The controller 23 can includes a memory 231 and a processor 232, as a hardware configuration. For example, the memory 231 can include a read only memory (ROM) configured to store various computer programs, and a random access memory (RAM) having a plurality of work areas in which various computer programs to be executed by the processor 232 are stored. The processor 232 is, for example, a central processing unit (CPU), and is configured to load a computer program specified from various computer programs incorporated in the ROM on the RAM and execute various processes in cooperation with the RAM.

The computer program may be stored in, for example, various types of non-transitory computer readable storage medium and supplied to the computer. The non-transitory computer readable storage medium includes various types of tangible storage media. Examples of the non-transitory computer readable medium include a magnetic recording medium (for example, a flexible disk, a magnetic tape, or a hard disk drive), a magneto-optical recording medium (for example, a magneto-optical disk), a CD-read only memory (ROM), a CD-R, a CD-R/W, and a semi-conductor memory (for example, a mask ROM, a programmable ROM (PROM), an erasable PROM (EPROM), or a flash ROM).

The controller 23 is configured to classify the physiological information input to the input interface 21 into a first physiological information group including physiological information during at least a first period and a second physiological information group including physiological information during a second period different from the first period. In the present embodiment, the first period is a period temporally later than the second period. However, the first period may be a period temporally earlier than the second period. Also in the present embodiment, it is assumed that a current time is 0:00 on Apr. 4, 2023. For example, in a case where the first period is a period from 0:00 on Apr. 3, 2023 to 23:59 on the same day and the second period is a period from 0:00 on Apr. 2, 2023 to 23:59 on the same day, the controller 23 classifies the physiological information during the period from 0:00 on Apr. 2, 2023 to 23:59 on Apr. 3, 2023 into a first physiological information group including physiological information on Apr. 3, 2023 and a second physiological information group including physiological information on Apr. 2, 2023. As described above, in the present embodiment, 24 hours (one day) immediately before the current time is set as the first period, and 24 hours (one day) immediately before the first period is set as the second period. The first period and the second period may have the same length or may have different lengths. The first period and the second period are not limited to a unit of a day. For example, the first period and the second period may be a unit of a month or a unit of predetermined time.

The controller 23 is configured to generate display data including the first physiological information group and the second physiological information group, and a first line AX1 (see FIG. 3) that is a timeline and a second line AX2 (see FIG. 3) that indicates a value related to the physiological information, based on the physiological information input to the input interface 21. For example, in a case where SpO2 is 98%, the value related to the physiological information is a value related to the physiological information (SpO2 in this example) at 98%. For example, in a case where the respiration rate is 20 times/min, 20 times/min is a value related to the physiological information (in this example, the respiration rate). That is, the value related to the physiological information is a size, a degree, or the like indicated by the physiological information.

The display data includes a first graph object G1 (see FIG. 3) indicating the size of each piece of physiological information included in the first physiological information group, and a second graph object G2 (see FIG. 3) indicating the size of each piece of physiological information included in the second physiological information group. The first graph object G1 and the second graph object G2 are, for example, polygonal line graphs, waveform graphs, bar graphs, or the like.

The controller 23 is configured to specify a first portion P1 (see FIG. 3) surrounded by the first graph object G1 and the first line AX1. The controller 23 is configured to specify a second portion P2 (see FIG. 3) surrounded by the second graph object G2 and the first line AX1. The controller 23 is configured to specify an overlapped portion P10 (see FIG. 3) which is a portion where the first portion P1 and the second portion P2 overlap. The controller 23 is configured to specify non-overlapped portions P20 and P30 (see FIG. 3) which is a portion where the first portion P1 and the second portion P2 do not overlap. A display mode of the overlapped portion P10 is different from display modes of the non-overlapped portions P20 and P30. The non-overlapped portions P20 and P30 are portions where the first portion P1 and the second portion P2 do not overlap, and correspond to portions indicating the difference between the first physiological information group and the second physiological information group.

The non-overlapped portion P20 in the first portion P1 is colored with a first color. The non-overlapped portion P30 in the second portion P2 is colored with a second color different from the first color. The second color is preferably a color opposite to the first color so that the non-overlapped portion P20 and the non-overlapped portion P30 can be easily distinguished from each other. For example, the first color is blue and the second color is red. The overlapped portion P10 is colored with a fourth color different from the first color and the second color. The fourth color may be a color related to both the first color and the second color (for example, a color in which the first color and the second color are combined) so that the user can intuitively recognize a portion on which the first portion P1 and the second portion P2 overlap each other. For example, when the first color is blue and the second color is red, the fourth color may be purple.

The first color, the second color, and the fourth color are specified by, for example, controller 23 adjusting RGB gradation. Note that RGB is so-called three primary colors of light, and R represents red, G represents green, and B represents blue. For example, the controller 23 may set an average value of the gradation of each color of RGB in the first color and the gradation of each color of RGB in the second color as the gradation of each color of RGB in the fourth color. In the present embodiment, the numbers below the decimal point is rounded up, but the numbers below the decimal point may be rounded down or may be rounded off. For example, in a case where the gradation of each color of RGB in the first color (blue) is (0, 0, 255) and the gradation of each color of RGB in the second color (red) is (255, 0, 0), the gradation of each color of RGB in the fourth color (purple) is (128, 0, 128). The gradation of each color of RGB in the first color, the second color, and the fourth color is not limited to this example. For example, when the first color is blue, the gradation of each color of RGB in the first color may be (1, 0, 255). The first color, the second color, and the fourth color may be specified using, for example, an xy chromaticity diagram.

The display unit 24 is, for example, a touch screen display such as a liquid crystal display or an organic EL display. The display unit 24 is configured to display, for example, display data received from the controller 23.

The output interface 25 is configured to output, to the terminal device 40, the display data and the like received from the controller 23. For example, the output interface 25 is configured to output, to the terminal device 40, an output signal corresponding to display data or the like. The output interface 25 may include a circuit configured to convert output data into an output signal that can be processed by the terminal device 40, as necessary.

The server 30 is, for example, a server computer on a cloud network. The server 30 can include a memory 31 and a controller 32.

The memory 31 may include, for example, a storage device (storage) such as a hard disk drive (HDD), a solid state drive (SSD), or a flash memory. The memory 31 is configured to store physiological information, previous history information, attribute information, condition information, administration information, and the like on the subject.

The controller 32 may have the same hardware configuration as the controller 23. For example, based on a request signal transmitted from the operation unit 22 of the information processing device 20, the controller 32 is configured to transmit, to the information processing device 20, all or some of the physiological information, previous history information, attribute information, condition information, administration information, and the like on the subject stored in the memory 31.

The terminal device 40 is, for example, an electronic device such as a desktop type PC, a notebook PC, a tablet terminal, or a smartphone. The terminal device 40 can include the display unit 41.

The display unit 41 is, for example, a touch screen display such as a liquid crystal display or an organic EL display. The display unit 41 is configured to display, for example, display data received from the controller 23.

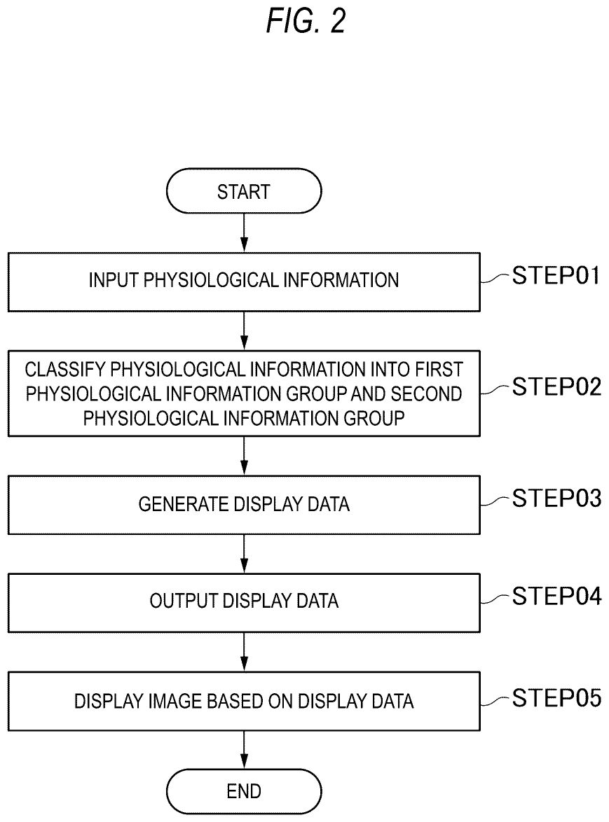

Next, an information processing method according to the first embodiment will be described with reference to FIGS. 2 and 3. FIG. 2 is a flowchart illustrating the information processing method related to generation of display data in the present embodiment. FIG. 3 is a diagram illustrating an image based on the display data. In the present embodiment, the controller 23 is configured to classify the physiological information input to the input interface 21 into the first physiological information group including the physiological information during the first period and the second physiological information group including the physiological information during the second period. The physiological information in the present embodiment is a respiration rate. In the present embodiment, the first period is the period from 0:00 on Apr. 3, 2023 to 23:59 on the same day, and the second period is the period from 0:00 on Apr. 2, 2023 to 23:59 on the same day. That is, in the present embodiment, the first period and the second period have the same length.

As illustrated in FIG. 2, the physiological information measured by the sensor 10 is input to the input interface 21 of the information processing device 20 (STEP01). In the present embodiment, the physiological information measured by the sensor 10 during the period from 0:00 on Apr. 2, 2023 to 23:59 on Apr. 3, 2023 is sequentially input to the input interface 21 every processing unit time as time elapses. The processing unit time is, for example, one hour, one minute, or one second. The input interface 21 is configured to transmit the input physiological information to the controller 23 or the server 30. The controller 23 receives the physiological information from the input interface 21, or the server 30 transmits the physiological information to the information processing device 20 based on a request signal from the operation unit 22, thereby obtaining the physiological information input to the input interface 21.

The controller 23 classifies the physiological information input to the input interface 21 into the first physiological information group including the physiological information during the first period and the second physiological information group including the physiological information during the second period (STEP02). In the present embodiment, the controller 23 classifies the physiological information input to the input interface 21 into the first physiological information group including physiological information on Apr. 3, 2023 and the second physiological information group including physiological information on Apr. 2, 2023.

The controller 23 generates the display data including the first physiological information group, the second physiological information group, the first line AX1, and the second line AX2 (STEP03). The display data is generated in a form in which the first physiological information group and the second physiological information group are displayed in a state where the first lines AX1 overlap each other and the second line AX2 is common.

The output interface 25 outputs the display data received from the controller 23 to the terminal device 40 (STEP04). In a case where the display data is output to the terminal device 40, an image 100 illustrated in FIG. 3 is displayed on the display unit 41 of the terminal device 40 (STEP05).

Here, the image 100 based on the display data generated by the controller 23 will be described with reference to FIG. 3. As illustrated in FIG. 3, the image 100 includes the first line AX1, the second line AX2, the first graph object G1, and the second graph object G2. The first line AX1 is a timeline and extends in a lateral direction (the left-right direction in FIG. 3). The second line AX2 indicates a value related to the physiological information and extends in a vertical direction (the up-down direction in FIG. 3). The first graph object G1 and the second graph object G2 are polygonal line graphs indicating respiration rates.

In the present embodiment, the first physiological information group and the second physiological information group are displayed in a state where a first displayable region R1 in which the first physiological information group is displayable and a second displayable region R2 in which the second physiological information group is displayable overlap each other on the same timeline (that is, on the first line AX1). In the present embodiment, the first displayable region R1 and the second displayable region R2 are the same region. The first graph object G1 is displayed in the first displayable region R1, and the second graph object G2 is displayed in the second displayable region R2. In other words, the first physiological information group and the second physiological information group are displayed in a state where the first lines AX1 overlap each other and the second line AX2 is common.

The controller 23 is configured to specify the overlapped portion P10 which is a portion where the first portion P1 surrounded by the first graph object G1 and the first line AX1, and the second portion P2 surrounded by the second graph object G2 and the first line AX1 overlap. The controller 23 is configured to specify the non-overlapped portions P20 and P30 which are portions where the first portion P1 and the second portion P2 do not overlap. The controller 23 is configured to set colors to be colored in the non-overlapped portions P20 and P30 and the overlapped portion P10 by adjusting the RGB gradation. In the present embodiment, the non-overlapped portion P20 (a portion of the first portion P1) is colored with blue (an example of the first color). The non-overlapped portion P30 (a portion of the second portion P2) is colored with red (an example of the second color). The overlapped portion P10 is colored with purple (an example of the fourth color) that is a combination of blue and red. Note that, in FIG. 3, a hatched portion with diagonal lines downward to the right indicates the portion colored with blue. A dotted hatched portion indicates the portion colored with red. A hatched portion with diagonal lines upward to the right indicates the portion colored with purple. In this way, the color (blue) colored in the non-overlapped portion P20 is the opposite color to the color (red) colored in the non-overlapped portion P30, and the non-overlapped portion P20 and the non-overlapped portion P30 are displayed in a form that can be easily distinguished from each other when viewed from the user. Since the display mode of the overlapped portion P10 is different from the display mode of the non-overlapped portions P20 and P30 (portions including only the first portion P1 or the second portion P2), the user can easily distinguish the overlapped portion P10 from the non-overlapped portions P20 and P30.

As illustrated in FIG. 3, an average value of values indicated by the physiological information (in the present embodiment, the respiration rate) included in the first physiological information group is displayed in a lower center of the image 100. Therefore, in the present embodiment, the numeral “21” indicating the average value is displayed in the lower center of the image 100. In the image 100, a location where the average value of the values indicated by the physiological information included in the first physiological information group is displayed is not limited to the lower center of the image 100. The value displayed in the lower center of the image 100 may be, for example, a value indicated by physiological information at any time during the first period.

In the medical field, it is required to quickly notice a condition change of a subject and perform an appropriate treatment before the condition of the subject deteriorates. On the other hand, the condition of the subject often changes rapidly, but even in such a case, the condition of the subject often changes gradually before the condition of the subject changes rapidly. For this reason, it is preferable that it is possible to recognize early that the condition of the subject changes gradually.

The information processing device 20 according to the above configuration is configured to generate the display data including the first physiological information group during the first period, the second physiological information group during the second period, the first line AX1 that is the timeline, and the second line AX2 that indicates a magnitude of the value of the physiological information on the subject. The display data is generated in the form in which the first physiological information group and the second physiological information group are displayed in a state where the first lines AX1 overlap each other and the second line AX2 is common. Therefore, the user can easily notice the difference between the first physiological information group during the first period and the second physiological information group during the second period by using the generated display data. As a result, the user can notice a slight change in the physiological information by using the display data, for example, even when the condition of the subject changes gradually. Accordingly, according to the information processing device 20, it is possible to support a medical worker to quickly notice a condition change of a subject.

In the information processing device 20 according to the above configuration, the display mode of the overlapped portion P10, which is a portion where the first portion P1 surrounded by the first graph object G1 and the first line AX1 and the second portion P2 surrounded by the second graph object G2 and the first line AX1 overlap is different from the display modes of the non-overlapped portions P20 and P30 which are portions where the first portion P1 and the second portion P2 do not overlap. The non-overlapped portions P20 and P30 are portions indicating the difference between the first physiological information group and the second physiological information group. Since the display mode of the non-overlapped portions P20 and P30 is different from the display mode of the overlapped portion P10, it is easy to visually understand the difference between the first physiological information group and the second physiological information group.

In the information processing device 20 according to the above configuration, the overlapped portion P10 is colored with a color obtained by combining the first color colored in the non-overlapped portion P20 in the first portion P1 and the second color colored in the non-overlapped portion P30 in the second portion P2. Therefore, the user can easily distinguish and recognize the overlapped portion P10 and the non-overlapped portions P20 and P30.

### Second Embodiment

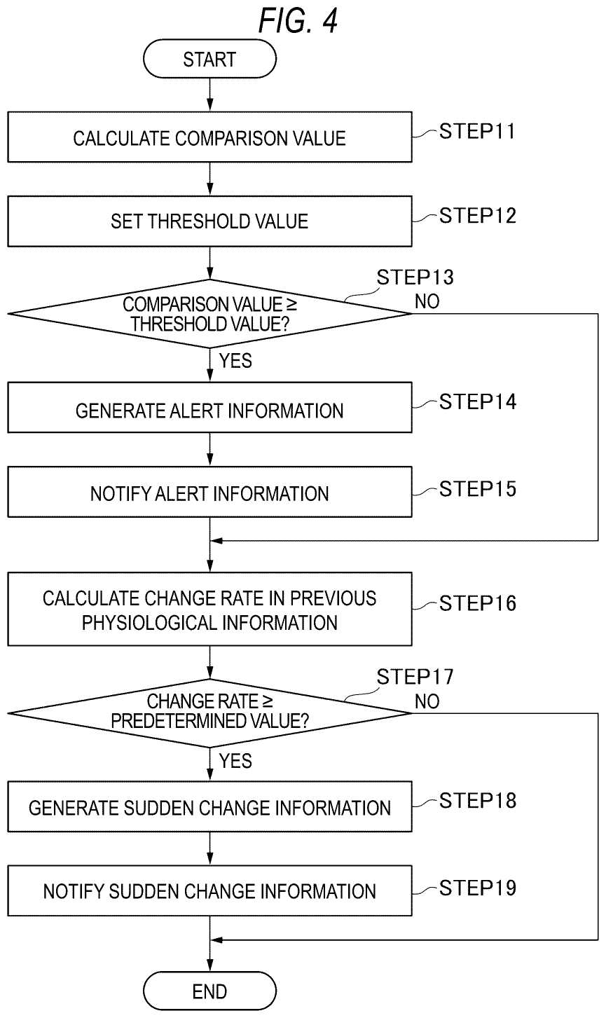

Next, an information processing system 1A according to a second embodiment will be described with reference to FIGS. 1 to 4. In the second embodiment, portions similar to those of the first embodiment will be described using the same reference numerals as those of the first embodiment, and description of overlapped portions will be appropriately omitted. As illustrated by a broken line in FIG. 1, the information processing system 1A includes an information processing device 20A including a notification unit 27. In this respect, the information processing system 1A according to the present embodiment is different from the information processing system 1 according to the first embodiment. The present embodiment is different from the first embodiment in that the information processing device 20 executes not only the information processing method related to the generation of the display data illustrated in FIG. 2 but also an information processing method related to generation of alert information and sudden change information illustrated in FIG. 4.

Also in the present embodiment, it is assumed that the current time is 0:00 on Apr. 4, 2023. Also in the present embodiment, the first period is the period from 0:00 on Apr. 3, 2023 to 23:59 on the same day, and the second period is the period from 0:00 on Apr. 2, 2023 to 23:59 on the same day. That is, also in the present embodiment, the first period is a period temporally later than the second period. The first period and the second period have the same length. Further, in the present embodiment, the physiological information included in the first physiological information group and the physiological information included in the second physiological information group are the same as the physiological information included in the first physiological information group and the physiological information included in the second physiological information group in the first embodiment. That is, also in the present embodiment, the image based on the display data generated by the controller 23 is the image 100 illustrated in FIG. 3.

The controller 23 is configured to calculate a comparison value between a first value based on the first physiological information group and a second value based on the second physiological information group. The comparison value may be, for example, a value calculated by summing up the differences, the differences being differences between values indicated by physiological information at respective times in the first physiological information group and the second physiological information group for each time. The difference between the values indicated by the physiological information at the respective times in the first physiological information group and the second physiological information group is a value obtained by subtracting the value indicated by the physiological information included in the second physiological information group at the same time from the value indicated by the physiological information included in the first physiological information group at a certain time. The comparison value may be, for example, a value obtained by subtracting the total value (a second total value) of the values indicated by the pieces of physiological information included in the second physiological information group from the total value (a first total value) of the values indicated by the pieces of physiological information included in the first physiological information group. The comparison value may be, for example, a value obtained by dividing the first total value by the second total value. The comparison value may be, for example, a value obtained by subtracting an average value (a second average value) of the values indicated by the pieces of physiological information included in the second physiological information group from an average value (a first average value) of the values indicated by the pieces of physiological information included in the first physiological information group. The comparison value may be, for example, a value obtained by dividing the first average value by the second average value. The comparison value may be, for example, a value obtained by subtracting a central value (a second central value) of each piece of physiological information included in the second physiological information group from a central value (a first central value) of each piece of physiological information included in the first physiological information group. The comparison value may be, for example, a value obtained by dividing the first central value by the second central value. The comparison value may be, for example, the greatest difference (the maximum difference) among the differences between the values indicated by the pieces of physiological information at the respective times in the first physiological information group and the second physiological information group.

The controller 23 is configured to determine whether the calculated comparison value is equal to or greater than a threshold value. In a case where it is determined that the calculated comparison value is equal to or greater than the threshold value, the controller 23 generates the alert information for prompting confirmation of the condition of the subject. The threshold value is set based on, for example, a reference value or the like for distinguishing between a normal value and an abnormal value defined by a medical guideline or the like for each physiological information. For example, the controller 23 is configured to set a threshold value related to the generation of the alert information from a change tendency of the first physiological information group. For example, in a case where the physiological information is a respiration rate, a respiration rate of 12 times/min or more and less than 21 times/min is a normal value, and a respiration rate of less than 12 times/min or 21 times/min or more is an abnormal value. For example, in a case where the respiration rate of the subject increases with the elapse of time during the first period, the change tendency of the first physiological information group is an upward tendency, and the controller 23 sets the threshold value based on an upper limit reference value (21 times/minute). The threshold value in this case is, for example, +1440 times/day. On the other hand, for example, in a case where the respiration rate of the subject decreases with the elapse of time during the first period, the change tendency of the first physiological information group is a downward tendency, and the controller 23 sets the threshold value based on a lower limit reference value (12 times/minute). The threshold value in this case is, for example, −1440 times/day. In this manner, the controller 23 can set the threshold value related to the generation of the alert information after determining whether the value indicated by the physiological information included in the first physiological information group increases with the elapse of time or decreases with the elapse of time.

For example, the controller 23 may be configured to set the threshold value related to the generation of the alert information, based on at least one of previous history information, attribute information, condition information, or administration information of the subject. The previous history information is, for example, information indicating a disease that has been caused by the subject so far, treatment or surgery performed for the disease, or the like. The attribute information is information indicating the gender of the subject, the age of the subject, the ward where the subject is located, and the like. The condition information is information indicating the condition of the subject. The administration information is information indicating a drug or the like administered to the subject. For example, the controller 23 is configured to set the threshold value based on the upper limit reference value (21 times/minute) related to the respiration rate, based on the condition information indicating that the subject is pneumonia. This is because in a case where the subject is pneumonia, the subject is often in a state of tachypnea. The controller 23 is configured to transmit the generated alert information to the notification unit 27.

The controller 23 is configured to calculate a change value of the previous physiological information. The previous physiological information is physiological information measured at a time close to the current time (for example, a time several seconds to several minutes before the current time). The change value is, for example, a change rate or a variation.

Here, the change rate in the previous physiological information will be described. Assuming that a value indicated by physiological information measured at a time one minute before the current time is A and a value indicated by physiological information measured at a time two minutes before the current time is B, the controller 23 calculates the change rate X based on the following equation (1). In the present embodiment, the numbers below the decimal point is rounded up, but the numbers below the decimal point may be rounded down or may be rounded off. X (%)=A/B*100

In a case where the respiration rate indicated by the physiological information at the time (23:59 on Apr. 3, 2023) one minute before the current time is 26 times and the respiration rate indicated by the physiological information at the time (23:58 on Apr. 3, 2023) two minutes before the current time is 22 times, the change rate is 119%.

Next, the variation in the previous physiological information will be described. The controller 23 is configured to subtract the value indicated by the physiological information measured at the time two minutes before the current time from the value indicated by the physiological information measured at the time one minute before the current time, to calculate the variation of the previous physiological information. In a case where the respiration rate indicated by the physiological information at the time (23:59 on Apr. 3, 2023) one minute before the current time is 26 times and the respiration rate indicated by the physiological information at the time (23:58 on Apr. 3, 2023) two minutes before the current time is 22 times, the variation is 4.

The controller 23 is configured to determine whether the change value of the previous physiological information is equal to or greater than a predetermined value. In a case where it is determined that the change value of the previous physiological information is equal to or greater than the predetermined value, the controller 23 generates the sudden change information indicating that the condition of the subject has suddenly changed. When the change rate is adopted as the change value, the predetermined value is, for example, 110%. However, the predetermined value is not limited to 110%. When the variation is adopted as the change value, the predetermined value is, for example, 4. However, the predetermined value is not limited to 4. The controller 23 is configured to transmit the generated sudden change information to the notification unit 27.

The notification unit 27 is configured to notify the medical worker or the like of the alert information and the sudden change information received from the controller 23. The notification performed by the notification unit 27 includes at least one of visual notification, auditory notification, or tactile notification. For example, when the notification performed by the notification unit 27 is auditory notification, the notification unit 27 is a speaker or the like.

Next, the information processing method according to the second embodiment will be described with reference to FIG. 4. In the present embodiment, the controller 23 is configured to calculate a difference between the values indicated by the physiological information at each time in the first physiological information group and the second physiological information group for each time, and sum up the differences calculated for the respective times to calculate the comparison value. In the present embodiment, the controller 23 is configured to calculate the change rate in the previous physiological information as the change value of the previous physiological information. First, the information processing device 20 is configured to execute the information processing method related to the generation of the display data illustrated in FIG. 2. The information processing method related to the generation of the display data executed in the present embodiment is the same as the information processing method related to the generation of the display data executed in the first embodiment.

The information processing device 20 is configured to execute the information processing method related to the generation of the alert information and the sudden change information illustrated in FIG. 4. In the present embodiment, the information processing method related to the generation of the alert information and the sudden change information is executed after the information processing method related to the generation of the display data. However, the information processing method related to the generation of the alert information and the sudden change information may be executed simultaneously with the information processing method related to the generation of the display data, or may be executed before the information processing method related to the generation of the display data.

As illustrated in FIG. 4, the controller 23 calculates the comparison value between the first value based on the first physiological information group and the second value based on the second physiological information group (STEP11). For example, as illustrated in FIG. 3, the respiration rate at 12:00 on Apr. 2, 2023 is 19 times/min, and the respiration rate at 12:00 on Apr. 3, 2023 is 13 times/min. In accordance with this, the controller 23 determines that the difference between 12:00 on Apr. 2, 2023 and Apr. 3, 2023 is −6. The controller 23 calculates a comparison value by performing this calculation for each time, and summing up the differences calculated for each time. In the present embodiment, the comparison value calculated by the controller 23 is +1500 times/day.

As illustrated in FIG. 4, the controller 23 sets the threshold value related to the generation of the alert information based on the change tendency of the first physiological information group (STEP12). As illustrated in FIG. 3, during the first period (in particular, 20:30 on Apr. 3, 2023), the respiration rate of the subject increases with the elapse of time, and the change tendency of the first physiological information group is an upward tendency. In accordance with this, the controller 23 sets the threshold value related to the generation of the alert information based on the upper limit reference value related to the respiration rate. In the present embodiment, the threshold value is +1440 times/day.

As illustrated in FIG. 4, the controller 23 determines whether the calculated comparison value is equal to or greater than the set threshold value related to the generation of the alert information (STEP13). In a case where the calculated comparison value is equal to or greater than the set threshold value related to the generation of the alert information (YES in STEP13), the controller 23 generates the alert information (STEP14). After generating the alert information, the controller 23 performs STEP15. On the other hand, in a case where the calculated comparison value is less than the set threshold value related to the generation of the alert information (NO in STEP13), the controller 23 performs STEP16. In the present embodiment, the comparison value is +1500 times/day and is equal to or greater than the threshold value (+1440 times/day) related to the generation of the alert information (YES in STEP13), and the controller 23 generates the alert information (STEP14).

The controller 23 causes the notification unit 27 to notify the alert information by transmitting the generated alert information to the notification unit 27 (STEP15). The notification unit 27 issues, for example, a voice message of “Please confirm the condition of the person A.”. However, the notification unit 27 may notify the alert information by causing the display unit 24 of the information processing device 20 or the display unit 41 of the terminal device 40 to display a text message of “Please confirm the condition of the person A.”.

The controller 23 calculates the change rate in the previous physiological information (STEP16). In the present embodiment, since the current time is 0:00 on Apr. 4, 2023, as illustrated in FIG. 3, the respiration rate indicated by the physiological information at the time (23:59 on Apr. 3, 2023) one minute before the current time is 26 times. The respiration rate indicated by the physiological information at a time (23:58 on Apr. 3, 2023) two minutes before the current time is 22. According to STEP15, the change rate in the physiological information immediately before calculated by the controller 23 is 119%.

The controller 23 determines whether the calculated change rate in the immediately previous physiological information is equal to or greater than the predetermined value (STEP17). In a case where the calculated change rate in the immediately previous physiological information is equal to or greater than the predetermined value (YES in STEP17), the controller 23 generates the sudden change information (STEP18). After generating the sudden change information, the controller 23 performs STEP19. On the other hand, in a case where the calculated change rate in the immediately previous physiological information is less than the predetermined value (NO in STEP17), the controller 23 ends the processing related to the information processing method related to the generation of the alert information and the sudden change information. In the present embodiment, the change rate (119%) in the previous physiological information calculated by the controller 23 is equal to or greater than the predetermined value (110%) (YES in STEP17), and the controller 23 generates the sudden change information (STEP18).

The controller 23 causes the notification unit 27 to notify the sudden change information by transmitting the generated sudden change information to the notification unit 27 (STEP19). The notification unit 27 issues, for example, a voice message of “The condition of the person A suddenly changes.”. However, the notification unit 27 may notify the sudden change information by causing the display unit 24 of the information processing device 20 or the display unit 41 of the terminal device 40 to display a text message of “The condition of the person A suddenly changes.”.

The information processing device 20A according to the above configuration also achieves the same effects as the information processing device 20 according to the first embodiment.

According to the information processing device 20A according to the above configuration, in a case where the comparison value between the first value based on the first physiological information group and the second value based on the second physiological information group is equal to or greater than the threshold value, the alert information for prompting confirmation of the condition of the subject is generated. For example, by transmitting such alert information to the medical worker, it is possible to cause the medical worker to reliably confirm the condition of the subject.

According to the information processing device 20A according to the above configuration, since the threshold value related to the comparison value based on the change tendency of the first physiological information group during the first period which is a period temporally later than the second period, it is possible to generate appropriate alert information according to the tendency of the condition change of the subject.

According to the information processing device 20A according to the above configuration, in a case where the change rate in the previous physiological information is equal to or greater than the predetermined value, the sudden change information indicating that the condition of the subject has suddenly changed is generated. For example, by using such sudden change information, a medical worker can notice a sudden change in the condition of the subject.

### First Modification of First Embodiment

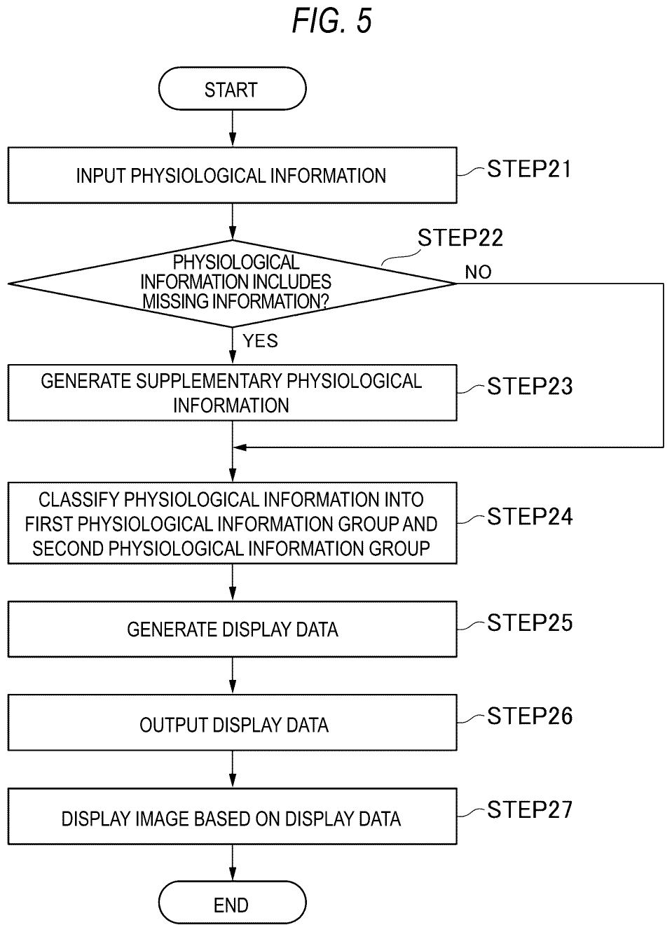

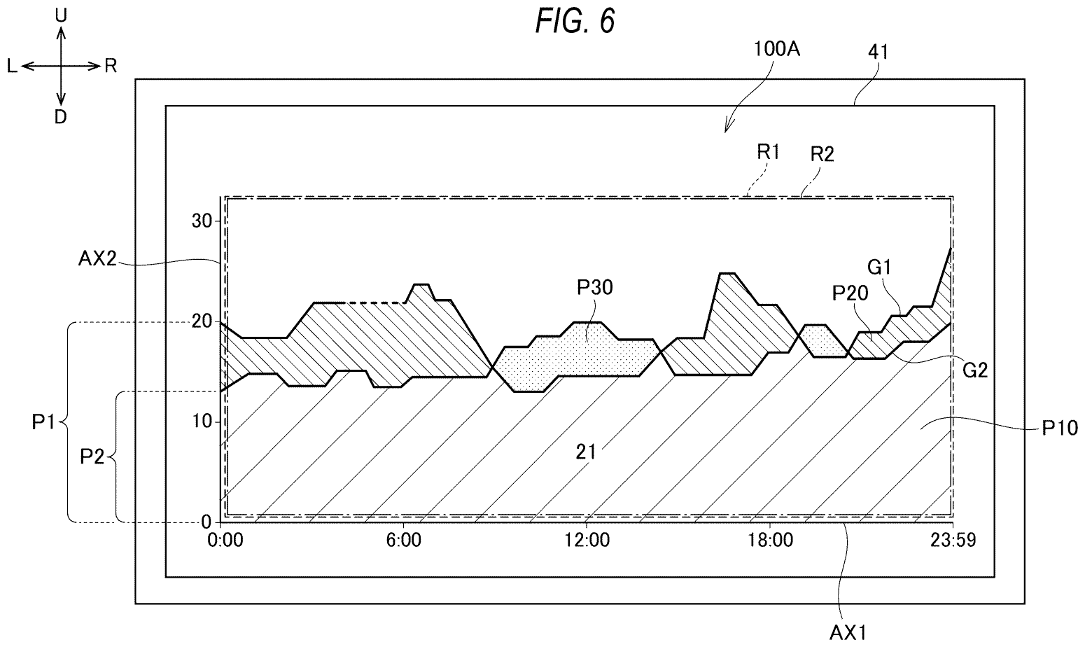

Next, a first modification of the first embodiment will be described with reference to FIGS. 5 and 6. In the present modification, portions similar to those of the first embodiment will be described using the same reference numerals as those of the first embodiment, and description of overlapped portions will be appropriately omitted. The present modification is different from the first embodiment in that, in a case where physiological information is missing during at least a portion of the first period, the controller 23 generates supplementary physiological information for compensating for missing physiological information based on the physiological information input to the input interface 21. The supplementary physiological information is temporary physiological information for compensating missing physiological information. In the present modification, it is assumed that the physiological information at 4:00 on Apr. 3, 2023 to 6:00 on the same day is missing.

Also in the present modification, the first period is the period from 0:00 on Apr. 3, 2023 to 23:59 on the same day, and the second period is the period from 0:00 on Apr. 2, 2023 to 23:59 on the same day. That is, also in the present modification, the first period is a period temporally later than the second period. However, the first period may be a period temporally earlier than the second period. The first period and the second period have the same length. However, the first period and the second period may have different lengths.

In a case where there is a time at which the physiological information on the subject cannot be measured, the sensor 10 inputs, to the input interface 21, physiological information including missing information indicating that the physiological information is missing. The missing information is, for example, information simply indicating that there is no physiological information, error information generated in response to the sensor 10 being unable to measure physiological information, or the like.

The controller 23 is configured to determine whether the physiological information input to the input interface 21 includes the missing information. In a case where it is determined that the missing information is included in the physiological information input to the input interface 21, the controller 23 generates the supplementary physiological information for compensating for the missing physiological information based on the physiological information input to the input interface 21. In a case where the supplementary physiological information is generated, the controller 23 includes the generated supplementary physiological information in the physiological information and classifies the physiological information into the first physiological information group and the second physiological information group.

Next, an information processing method according to the present modification will be described with reference to FIGS. 5 and 6. FIG. 5 is a flowchart illustrating the information processing method related to the generation of the display data in the present modification.

STEP21 is the same as STEP01 of the first embodiment. However, in the present modification, the sensor 10 cannot measure the physiological information from 4:00 on Apr. 3, 2023 to 6:00 on the same day. Therefore, the sensor 10 inputs, to the input interface 21, the physiological information including the missing information indicating that the physiological information is missing from 4:00 on Apr. 3, 2023 to 6:00 on the same day (an example of at least a portion of the first period).

The controller 23 determines whether the physiological information input to the input interface 21 includes the missing information (STEP22). In a case where it is determined that the missing information is included in the physiological information input to the input interface 21 (YES in STEP22), the controller 23 generates the supplementary physiological information (STEP23) and then performs STEP24. On the other hand, in a case where it is determined that the physiological information input to the input interface 21 does not include the missing information (NO in STEP22), the controller 23 performs STEP24. In the present modification, the physiological information input to the input interface 21 includes the missing information indicating that the physiological information is missing from 4:00 on Apr. 3, 2023 to 6:00 on the same day. Therefore, the controller 23 determines that the physiological information input to the input interface 21 includes the missing information (YES in STEP22), and generates the supplementary physiological information (STEP23). The supplementary physiological information generated at this time is temporary physiological information for compensating for the missing physiological information at 4:00 on Apr. 3, 2023 to 6:00 on the same day.

STEP24 to STEP27 are the same as STEP02 to STEP05 of the first embodiment. However, in the present modification, since the controller 23 generates the supplementary physiological information, the controller 23 includes the generated supplementary physiological information in the physiological information and classifies the physiological information into the first physiological information group and the second physiological information group in STEP24. In the present modification, the controller 23 generates the display data such that a display mode corresponding to the supplementary physiological information is different from a display mode corresponding to the physiological information input to the input interface 21.

Here, the display data generated by the controller 23 and an image 100A based on the display data will be described with reference to FIG. 6. In the present modification, since the physiological information is missing from 4:00 on Apr. 3, 2023 to 6:00 on the same day, the controller 23 generates display data in which a start point (a portion of 4:00 on the same day) and an end point (a portion of 6:00 on the same day) of the missing portion are connected by a straight line, for example. In FIG. 6, the straight line connecting the start point and the end point of the missing portion is drawn by a broken line, and the other lines are drawn by solid lines. However, a display mode of the straight line connecting the start point and the end point of the missing portion may be different from a display mode of the other lines, and is not limited to the broken line. As described above, the display mode corresponding to the supplementary physiological information is different from the display mode corresponding to the physiological information input to the input interface 21.

The information processing device 20 according to the present modification also achieves the same effect as the information processing device 20 according to the first embodiment.

According to the information processing device 20 according to the above configuration, even in a case where the physiological information is missing from 4:00 on Apr. 3, 2023 to 6:00 on the same day, the display data is generated after the supplementary physiological information for compensating for the missing physiological information is generated based on the physiological information input to the input interface 21. The display data generated at this time is generated in a form in which the first physiological information group and the second physiological information group are displayed in a state where the first lines AX1 overlap each other and the second line AX2 is common. Therefore, according to the information processing device 20, even in a case where the physiological information is missing, it is possible to support a medical worker to quickly notice the condition change of the subject.

According to the information processing device 20 having the above configuration, since the display mode corresponding to the supplementary physiological information is different from the display mode corresponding to the physiological information input to the input interface 21, the user such as a medical worker can easily distinguish the supplementary physiological information from the physiological information.

### Second Modification of First Embodiment

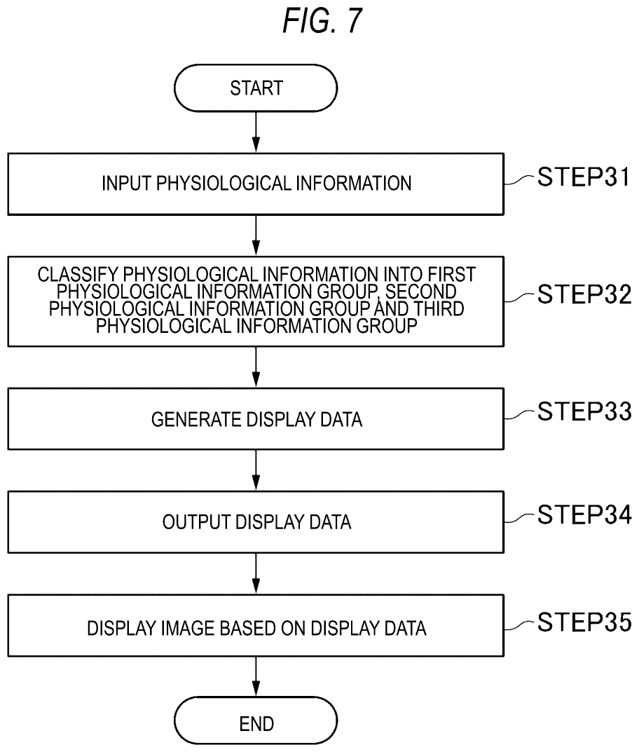

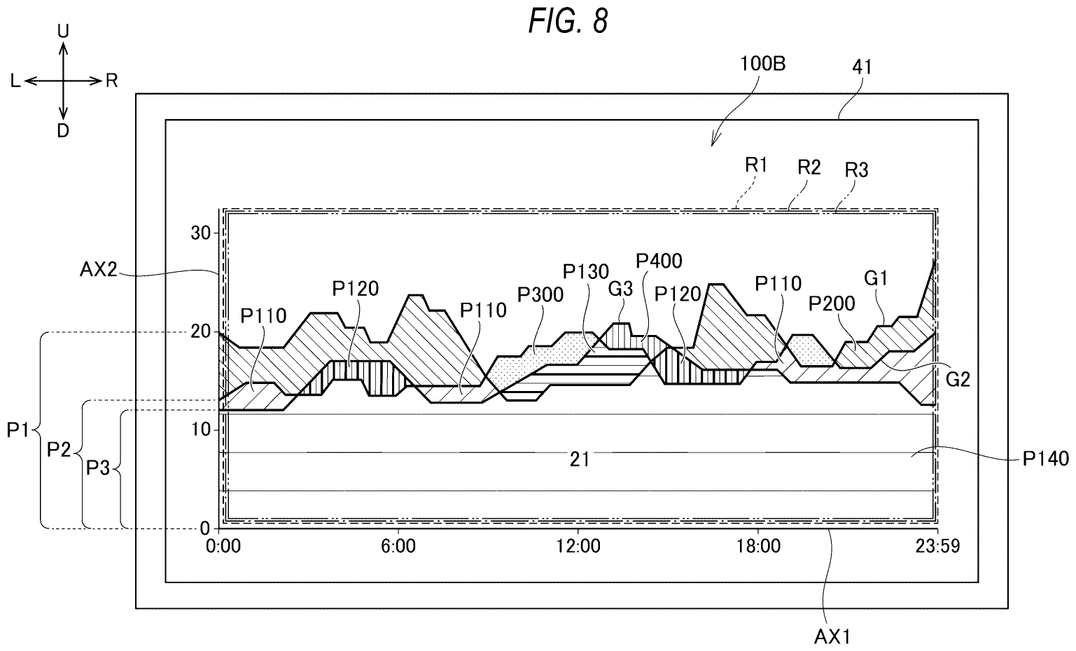

Next, a second modification of the first embodiment will be described with reference to FIGS. 7 and 8. In the present modification, portions similar to those of the first embodiment will be described using the same reference numerals as those of the first embodiment, and description of overlapped portions will be appropriately omitted. The present modification is different from the first embodiment in that the controller 23 classifies the physiological information input to the input interface 21 into the first physiological information group, the second physiological information group, and a third physiological information group. Therefore, in the present modification, an information processing method illustrated in FIG. 7 is executed instead of the information processing method illustrated in FIG. 2.

In the present modification, the sensor 10 is configured to measure the physiological information on the subject during a period from 0:00 on Apr. 1, 2023 to 23:59 on Apr. 3, 2023. The physiological information measured by the sensor 10 is input to the information processing device 20 as an electric signal.

The controller 23 is configured to classify the physiological information during the period from 0:00 on Apr. 1, 2023 to 23:59 on Apr. 3, 2023 into a first physiological information group including physiological information on Apr. 3, 2023, a second physiological information group including physiological information on Apr. 2, 2023, and a third physiological information group including physiological information on Apr. 1, 2023. Also in the present modification, the physiological information is the respiration rate. In the present modification, the first period is a period from 0:00 on Apr. 3, 2023 to 23:59 on the same day. The second period is a period from 0:00 on Apr. 2, 2023 to 23:59 on the same day. The third period is a period from 0:00 on Apr. 1, 2023 to 23:59 on the same day. That is, in the present modification, the first period, the second period, and the third period have the same length. However, the first period, the second period, and the third period may have different lengths.

The controller 23 is configured to generate display data including the first physiological information group, the second physiological information group, the third physiological information group, the first line AX1, and the second line AX2, based on the physiological information input to the input interface 21.

The display data includes the first graph object G1 and the second graph object G2 similar to those in the first embodiment, and a third graph object G3 (see FIG. 8) indicating the size of each piece of physiological information included in the third physiological information group. The third graph object G3 is, for example, a polygonal line graph, a waveform graph, or a bar graph.

The controller 23 is configured to specify the first portion P1 (see FIG. 8) surrounded by the first graph object G1 and the first line AX1. The controller 23 is configured to specify the second portion P2 (see FIG. 8) surrounded by the second graph object G2 and the first line AX1. The controller 23 is configured to specify a third portion P3 (see FIG. 8) surrounded by the third graph object G3 and the first line AX1. The controller 23 is configured to specify overlapped portions P110 (see FIG. 8) which are portions where the first portion P1 and the second portion P1 overlap. The controller 23 is configured to specify overlapped portions P120 (see FIG. 8) which are portions where the first portion P1 and the third portion P3 overlap. The controller 23 is configured to specify an overlapped portion P130 which is a portion where the second portion P2 and the third portion P3 overlap. The controller 23 is configured to specify an overlapped portion P140 which is a portion where the first portion P1, the second portion P2, and the third portion P3 overlap. The controller 23 is configured to specify non-overlapped portions P200, P300, and P400 (see FIG. 8) which are portions where at least two of the first portion P1, the second portion P2, and the third portion P3 do not overlap (that is, portions other than the overlapped portions P110, P120, P130, and P140).

The controller 23 is configured to color the non-overlapped portion P200 (a portion of the first portion P1), the non-overlapped portion P300 (a portion of the second portion P2), the non-overlapped portion P400 (a portion of the third portion P3), the overlapped portion P110, the overlapped portion P120, the overlapped portion P130, and the overlapped portion P140. Also in the present modification, the controller 23 is configured to specify colors to be colored in the non-overlapped portions P200, P300, and P400 and the overlapped portions P110, P120, P130, and P140 by adjusting the RGB gradation. However, the controller 23 may specify the colors to be colored in the non-overlapped portions P200, P300, and P400 and the overlapped portions P110, P120, P130, and P140 using an xy chromaticity diagram or the like.

The non-overlapped portion P200 in the first portion P1 is colored with the first color. In the present modification, the first color is blue. The non-overlapped portion P300 in the second portion P2 is colored with the second color. In the present modification, the second color is red. The non-overlapped portion P400 in the third portion P3 is colored with a third color. In the present modification, the third color is green. The overlapped portion P110 is colored with a color related to both the first color and the second color. The overlapped portion P110 is colored with purple, which is a combination of the first color and the second color, for example. The overlapped portion P120 is colored with a color related to both the first color and the third color. The overlapped portion P120 is colored with blue-green, which is a combination of the first color and the third color, for example. The overlapped portion P130 is colored with a color related to both the second color and the third color. The overlapped portion P130 is colored with yellow, which is a combination of the second color and the third color, for example. The overlapped portion P140 is colored with a color related to the first color, the second color, and the third color. The overlapped portion P140 is colored with gray, which is a combination of the first color, the second color, and the third color, for example. However, the colors of the non-overlapped portions P200, P300, and P400 and the overlapped portions P110, P120, P130, and P140 are not limited to this example.

Next, the information processing method according to the present modification will be described with reference to FIG. 7. FIG. 7 is a flowchart illustrating the information processing method related to the generation of the display data in the present modification.

STEP31 is the same as STEP01 of the first embodiment. However, in the present modification, the physiological information during the period from 0:00 on Apr. 1, 2023 to 23:59 on Apr. 3, 2023 is input to the input interface 21.

The controller 23 classifies the physiological information input to the input interface 21 into the first physiological information group including the physiological information during the first period, the second physiological information group including the physiological information during the second period, and the third physiological information group including the physiological information during the third period (STEP32). In the present modification, the controller 23 classifies the physiological information input to the input interface 21 into the first physiological information group including the physiological information on Apr. 3, 2023, the second physiological information group including the physiological information on Apr. 2, 2023, and the third physiological information group including the physiological information on Apr. 1, 2023.

STEP33 to STEP35 are the same as STEP03 to STEP05 of the first embodiment. However, in the present modification, in STEP33, the controller 23 generates the display data including the first physiological information group, the second physiological information group, the third physiological information group, the first line AX1, and the second line AX2. The display data is generated in a form in which the first physiological information group, the second physiological information group, and the third physiological information group are displayed in a state where the first lines AX1 overlap each other and the second line AX2 is common.

Here, the display data generated by the controller 23 and an image 100B based on the display data will be described with reference to FIG. 8. As illustrated in FIG. 8, the image 100B includes the first line AX1, the second line AX2, the first graph object G1, the second graph object G2, and the third graph object G3. The first graph object G1, the second graph object G2, and the third graph object G3 are polygonal line graphs indicating respiration rates.

In the present modification, the first physiological information group, the second physiological information group, and the third graph object G3 are displayed in a state where the first displayable region R1 in which the first physiological information group is displayable, the second displayable region R2 in which the second physiological information group is displayable, and a third displayable region R3 in which the third physiological information group is displayable overlap each other on the same timeline (that is, on the first line AX1). In the present modification, the first displayable region R1, the second displayable region R2, and the third displayable region R3 are the same region. The first graph object G1 is displayed in the first displayable region R1, the second graph object G2 is displayed in the second displayable region R2, and the third graph object G3 is displayed in the third displayable region R3. In other words, the first physiological information group, the second physiological information group, and the third physiological information group are displayed in a state where the first lines AX1 overlap each other and the second line AX2 is common.

In the present modification, the non-overlapped portion P200 is colored with blue (the example of the first color). The non-overlapped portion P300 is colored with red (the example of the second color). The non-overlapped portion P400 is colored with green (an example of the third color). The overlapped portion P110 is colored with purple (an example of the fourth color) that is a combination of blue and red. The overlapped portion P120 is colored with blue-green, which is a combination of blue and green. The overlapped portion P130 is colored with yellow, which is a combination of red and green. The overlapped portion P140 is colored with gray, which is a combination of blue, red, and green. Note that, in FIG. 8, a hatched portion with diagonal lines downward to the right indicates a portion colored with blue. A dotted hatched portion indicates the portion colored with red. A hatched portion of vertical lines indicates a portion colored with green. A hatched portion with diagonal lines upward to the right indicates the portion colored with purple. A hatched portion with vertical lines including a thin line and a thick line indicates a portion colored with blue-green. A hatched portion with horizontal lines including a thin line and a thick line indicates a portion colored with yellow. A hatched portion with a horizontal line indicates a portion colored with gray. As described above, also in the present modification, the display mode of the overlapped portions P110, P120, P130, and P140 is different from the display mode of the non-overlapped portions P200, P300, and P400 (portions including only the first portion P11, the second portion P12, and the third portion P13). Accordingly, the user can easily distinguish the overlapped portions P110, P120, P130, and P140 from the non-overlapped portions P200, P300, and P400.

### Third Modification of First Embodiment

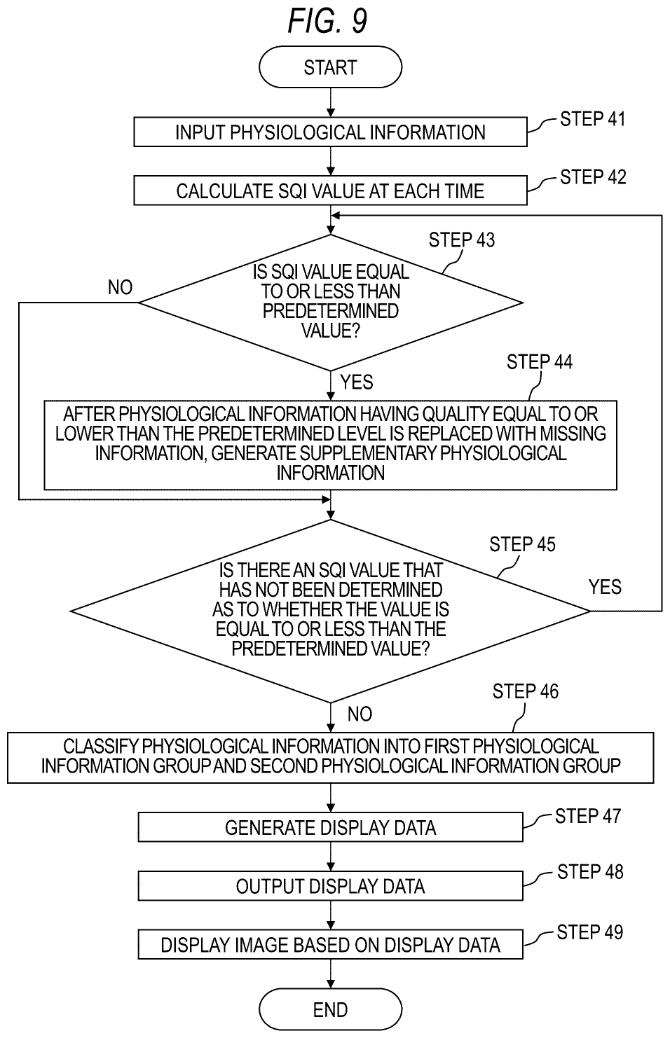

Next, a third modification of the first embodiment will be described with reference to FIG. 9. In the present modification, portions similar to those of the first embodiment will be described using the same reference numerals as those of the first embodiment, and description of overlapped portions will be appropriately omitted. The present modification is different from the first embodiment in that, in a case where quality of the physiological information during at least a portion of the first period is equal to or lower than a predetermined level, the controller 23 replaces the physiological information having quality equal to or lower than the predetermined level with the missing information and then generates the supplementary physiological information for compensating for the missing physiological information.

In the present modification, the controller 23 is configured to generate quality information indicating the quality of the physiological information, based on the physiological information and a quality reference value for evaluating the quality of the physiological information. In the present modification, the quality information is a signal quality indicator (SQI) value. However, the quality information is not limited to the SQI value. The SQI value is a numerical value obtained by quantifying the quality of the signal. The SQI value is obtained by, for example, comparing a value indicated by the physiological information with the quality reference value for evaluating the quality of the physiological information. The quality reference value is set based on, for example, a value that can be taken as a value indicated by the physiological information. For example, regarding the quality reference value related to the respiration rate, since the respiration rate is often 10 times/min or more and 30 times/min or less, the quality reference value related to the respiration rate is 20 times/min which is a central value of 10 times/min and 30 times/min. For example, in a case where the respiration rate is 5 times/min, the controller 23 calculates a value of 25% (5 times/20 times*100=25%) as the SQI value. For example, in a case where the respiration rate is 40 times/minute, the controller 23 calculates a value of 50% (20 times/40 times*100=50%) as the SQI value. Note that a method of calculating the SQI value is not limited to the above calculation method as long as the method is capable of calculating a value that indicates a degree of the difference between the value indicated by the measured physiological information and the quality reference value. The controller 23 is configured to determine whether the quality of the physiological information is equal to or lower than the predetermined level by determining whether the SQI value is equal to or less than a predetermined value. For example, the predetermined value related to the SQI is 50%. However, the predetermined value is not limited thereto. In a case where the SQI value is equal to or less than the predetermined value, the controller 23 determines that the quality of the physiological information is equal to or lower than the predetermined level.

The controller 23 is configured to calculate the SQI value at each time, based on the physiological information input to the input interface 21 and the quality reference value. In a case where it is determined that the quality of the physiological information is equal to or lower than the predetermined level, the controller 23 replaces the physiological information having the quality equal to or lower than the predetermined level with the missing information, and then generates the supplementary physiological information for compensating for the missing physiological information based on the physiological information input to the input interface 21. That is, the controller 23 is configured to discard physiological information whose quality is equal to or lower than the predetermined level, and includes the supplementary physiological information in the generated information instead of the discarded physiological information. The physiological information used when generating the supplementary physiological information is physiological information, among the physiological information input to the input interface 21, whose quality exceeding the predetermined level in the physiological information. In addition, in a case where the supplementary physiological information is generated, the controller 23 includes the generated supplementary physiological information in the physiological information and classifies the physiological information into the first physiological information group and the second physiological information group.

Next, an information processing method according to the present modification will be described with reference to FIG. 9. FIG. 9 is a flowchart illustrating the information processing method related to the generation of the display data in the present modification. Also in the present modification, the first period is the period from 0:00 on Apr. 3, 2023 to 23:59 on the same day, and the second period is the period from 0:00 on Apr. 2, 2023 to 23:59 on the same day. In the present modification, it is assumed that the quality of the physiological information at 4:00 on Apr. 3, 2023 to 6:00 on the same day is equal to or lower than the predetermined level, whereas the quality of the physiological information at other times exceeds the predetermined level.

STEP41 is the same as STEP01 of the first embodiment.

The controller 23 calculates the SQI value at each time based on the physiological information input to the input interface 21 and the quality reference value related to the physiological information (STEP42). That is, in STEP42, the controller 23 generates the quality information at each time based on the physiological information input to the input interface 21 and the quality reference value related to the physiological information.

The controller 23 determines whether the SQI value is equal to or less than the predetermined value (STEP43). In a case where the SQI value is equal to or less than the predetermined value (YES in STEP43), the controller 23 replaces the physiological information having the SQI value equal to or less than the predetermined value with the missing information, and then generates the supplementary physiological information for compensating for the missing physiological information based on the physiological information input to the input interface 21 (STEP44). After generating the supplementary physiological information, the controller 23 performs STEP45. On the other hand, in a case where the SQI value exceeds the predetermined value (NO in STEP43), the controller 23 performs STEP45 without generating the supplementary physiological information.

In the present modification, among the physiological information input to the input interface 21, the quality of the physiological information from 4:00 on Apr. 3, 2023 to 6:00 on the same day is equal to or lower than the predetermined level. Therefore, the controller 23 determines that the SQI value is equal to or less than the predetermined value for the physiological information at 4:00 on Apr. 3, 2023 to 6:00 on the same day (YES in STEP43), and generates the supplementary physiological information (STEP44). On the other hand, since the quality of the physiological information at the other time exceeds the predetermined level, the controller 23 determines that the SQI value exceeds the predetermined value for the physiological information at the other times (NO in STEP43), and does not generate the supplementary physiological information.

The controller 23 determines whether there is an SQI value that has not been determined as to whether the value is equal to or less than the predetermined value (STEP45). In a case where there is an SQI value that has not been determined as to whether the value is equal to or less than the predetermined value (YES in STEP45), the controller 23 executes STEP43. On the other hand, in a case where there is no SQI value that has not been determined as to whether the value is equal to or less than the predetermined value (NO in STEP45), that is, in a case where it is determined whether the SQI value is equal to or less than the predetermined value for the SQI values at all times, the controller 23 performs STEP46. In the present modification, for the SQI value for each time at 0:00 on Apr. 2, 2023 to 23:59 on April 3, when it is determined whether the SQI value is equal to or less than the predetermined value, the controller 23 performs STEP46.

STEP46 to STEP49 are the same as STEP02 to STEP05 of the first embodiment. However, in the present modification, since the controller 23 generates the supplementary physiological information, the controller 23 classifies the physiological information into the first physiological information group and the second physiological information group after including the supplementary physiological information generated in place of the physiological information having the SQI value equal to or less than the predetermined value (the physiological information at 4:00 on Apr. 3, 2023 to 6:00 on the same day) in the physiological information in STEP46. The physiological information measured from 4:00 on Apr. 3, 2023 to 6:00 on the same day is discarded. In the present modification, the controller 23 is configured to generate the display data such that the display mode corresponding to the supplementary physiological information is different from the display mode corresponding to the physiological information whose quality exceeds the predetermined level.

In the present modification, an image same as the image illustrated in FIG. 6 is displayed on the display unit 41 of the terminal device 40. In the present modification, the quality of physiological information at 4:00 on Apr. 3, 2023 to 6:00 on the same day is equal to or lower than the predetermined level. Therefore, for example, the controller 23 generates display data in which a start point (the portion of 4:00 on the same day) and an end point (the portion of 6:00 on the same day) of a portion where the quality of the physiological information is equal to or lower than the predetermined level are connected by a straight line. In the present modification, the straight line connecting the start point and the end point of the portion where the quality of the physiological information is equal to or lower than the predetermined level is drawn by a broken line, and the other lines are drawn by solid lines. However, the display mode of the straight line connecting the start point and the end point of the portion where the quality of the physiological information is equal to or lower than the predetermined level may be different from the display mode of the other lines, and is not limited to the broken line. As described above, the display mode corresponding to the supplementary physiological information is different from the display mode corresponding to the physiological information in a portion (that is, a portion where the quality of the physiological information exceeds the predetermined level) other than the portion from 4:00 on Apr. 3, 2023 to 6:00 on the same day.

The information processing device 20 according to the present modification also achieves the same effect as the information processing device 20 according to the first embodiment.

According to the information processing device 20 according to the above configuration, even if the quality of the physiological information from 4:00 on Apr. 3, 2023 to 6:00 on the same day is equal to or lower than the predetermined level, the display data is generated after the supplementary physiological information is generated. The display data generated at this time is generated in a form in which the first physiological information group and the second physiological information group are displayed in a state where the first lines AX1 overlap each other and the second line AX2 is common. Therefore, according to the information processing device 20, even in a case where the physiological information input to the input interface 21 includes the physiological information having the quality equal to or lower than the predetermined level, it is possible to support the medical worker to quickly notice the condition change of the subject.

According to the information processing device 20 having the above configuration, since the display mode corresponding to the supplementary physiological information is different from the display mode corresponding to the physiological information input to the input interface 21, the user such as a medical worker can easily distinguish the supplementary physiological information from the physiological information.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

In the second embodiment, the controller 23 is configured to generate the alert information and the sudden change information, but may be configured to generate only the alert information. That is, the controller 23 does not need to execute STEP16 to STEP19. The controller 23 may be configured to generate only the sudden change information. That is, the controller 23 may not execute STEP11 to STEP15.

In the second embodiment, the controller 23 sets the threshold value related to the comparison value based on the change tendency of the first physiological information group, but may set the threshold value related to the comparison value based on at least one of the previous history information, the attribute information, the condition information, and the administration information of the subject. In this case, since the controller 23 sets the threshold value related to the comparison value based on at least one of the previous history information, the attribute information, the condition information, and the administration information of the subject, appropriate alert information according to the characteristics of the subject can be generated.

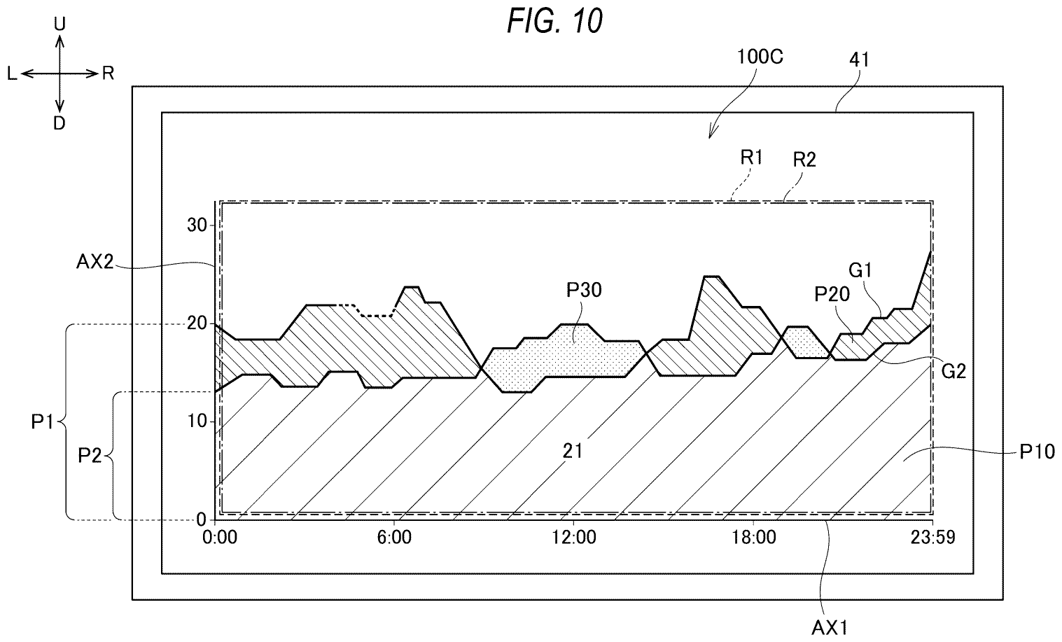

In the first modification of the first embodiment, the controller 23 is configured to generate the display data in which the start point (the portion at 4:00 on Apr. 3, 2023) and the end point (the portion at 6:00 on the same day) of the missing portion are connected by a straight line, but the present disclosure is not limited thereto. In the third modification of the first embodiment, the controller 23 is configured to generate the display data in which the start point (the portion at 4:00 on the same day) and the end point (the portion at 6:00 on the same day) of the portion where the quality of the physiological information is equal to or lower than the predetermined level are connected by a straight line, but the present disclosure is not limited thereto. For example, the controller 23 may estimate the change in the value of the physiological information from 4:00 on Apr. 3, 2023 to 6:00 on the same day based on the change in the value of the physiological information from 4:00 on Apr. 2, 2023 to 6:00 on the same day. That is, it may be estimated that the value of the physiological information from 4:00 on Apr. 3, 2023 to 6:00 on the same day changes in the same manner as the change in the value of the physiological information from 4:00 on Apr. 2, 2023 to 6:00 on the same day. In this case, an image 100C illustrated in FIG. 10 is displayed on the display unit 41 of the terminal device 40. Note that a line that continues from the start point to the end point of the missing portion or the portion where the quality of the physiological information is equal to or lower than the predetermined level is drawn by a broken line, and the other lines are drawn by solid lines. However, the display mode of the line continuing from the start point to the end point of the missing portion or the portion where the quality of the physiological information is equal to or lower than the predetermined level may be different from the display mode of the other lines, and is not limited to the broken line. As described above, also in this case, the display mode corresponding to the supplementary physiological information is different from the display mode corresponding to the physiological information input to the input interface 21.

In the above embodiments, the controller 23 is configured to classify the physiological information input to the input interface 21 into the first physiological information group and the second physiological information group, or the first physiological information group, the second physiological information group, and the third physiological information group, but the present disclosure is not limited thereto. For example, the controller 23 may be configured to classify the physiological information input to the input interface 21 into the first physiological information group, the second physiological information group, the third physiological information group, and a fourth physiological information group. That is, the controller 23 may be configured to classify the physiological information input to the input interface 21 into four or more physiological information groups.

In the above embodiments, the images 100, 100A, 100B, and 100C are displayed on the display unit 41 of the terminal device 40, but may be displayed on the display unit 24 of the information processing devices 20 and 20A, for example.

In the above embodiments, the information processing system 1 includes the sensor 10, the information processing device 20, the server 30, and the terminal device 40, but may include a central monitor instead of the server 30. In this case, the central monitor has the same function as the server 30.

In the above embodiments, the overlapped portions P10, P110, P120, P130, and P140 and the non-overlapped portions P20, P30, P200, P300, and P400 are colored with different colors, but the overlapped portions P10, P110, P120, P130, and P140 and the non-overlapped portions P20, P30, P200, P300, and P400 may not be colored. In this case, the display modes of the first graph object G1, the second graph object G2, and the third graph object G3 may be different. For example, the first graph object G1 may be a polygonal line graph having a solid line, the second graph object G2 may be a polygonal line graph having a broken line, and the third graph object G3 may be a one-dot chain polygonal line graph.

In the second embodiment, the comparison value is a value calculated by calculating differences between values indicated by physiological information at respective times in the first physiological information group and the second physiological information group for each time and summing up the differences, but the present disclosure is not limited thereto. The comparison value may be, for example, a value obtained by subtracting the second total value from the first total value, a value obtained by dividing the first total value by the second total value, a value obtained by subtracting the second average value from the first average value, a value obtained by dividing the first average value by the second average value, a value obtained by subtracting the second central value from the first central value, a value obtained by dividing the first central value by the second central value, or the maximum difference.

In the second embodiment, the controller 23 calculates the change rate in the previous physiological information, but may calculate the variation of the previous physiological information. In this case, when it is determined that the variation in the previous physiological information is equal to or greater than the predetermined value, the controller 23 generates the sudden change information.

As described above, the present specification discloses the following matters.

Clause 1. An information processing device including:
an input interface to which physiological information on a subject is input;
at least one processor; and
a memory configured to store at least one command that the processor is configured to execute,
in which in a case where the at least one command is executed by the processor, the information processing device is configured to:
   classifies the physiological information input to the input interface into a first physiological information group and a second physiological information group, the first physiological information group including the physiological information during at least a first period, the second physiological information group including the physiological information during a second period different from the first period; and
   generates display data including the first physiological information group, the second physiological information group, a first line that is a timeline, and a second line that indicates a value related to the physiological information; and
the display data is generated in a form in which the first physiological information group and the second physiological information group are displayed in a state where the first lines overlap each other and the second line is common.

Clause 2. The information processing device according to clause 1,
in which a display mode of an overlapped portion which is a portion where a first portion and a second portion overlap is different from a display mode of a non-overlapped portion which is a portion where the first portion and the second portion do not overlap, the first portion being surrounded by the first line and a first graph object indicating a size of each piece of the physiological information included in the first physiological information group, the second portion being surrounded by the first line and a second graph object indicating a size of each piece of the physiological information included in the second physiological information group.

Clause 3. The information processing device according to clause 1 or 2,
in which the first period and the second period have the same length,
the information processing device is configured to calculate a comparison value between a first value based on the first physiological information group and a second value based on the second physiological information group, and
in a case where the comparison value is equal to or greater than a threshold value, the information processing device is configured to generate alert information for prompting confirmation of a condition of the subject.

Clause 4. The information processing device according to clause 2,
in which the non-overlapped portion in the first portion is colored with a first color,
the non-overlapped portion in the second portion is colored with a second color different from the first color, and
the overlapped portion is colored with a color different from the first color and the second color.

Clause 5. The information processing device according to clause 3,
in which the first period is a period temporally later than the second period, and
the information processing device is configured to set the threshold value, based on a change tendency of the first physiological information group.

Clause 6. The information processing device according to clause 3,
in which the information processing device is configured to set the threshold value, based on at least one of previous history information, attribute information, condition information, or administration information of the subject.

Clause 7. The information processing device according to any one of clauses 1 to 6,
in which the information processing device is configured to:
   calculate a change value of the previous physiological information, and
   in a case where the change value is equal to or greater than a predetermined value, calculate sudden change information indicating that a condition of the subject has suddenly changed.

Clause 8. The information processing device according any one of clauses 1 to 7,
in which in a case where the first physiological information group includes missing information indicating that the physiological information is missing during at least a portion of the first period, the information processing device is configured to generate supplementary physiological information for compensating for the missing physiological information based on the physiological information input to the input interface.

Clause 9. The information processing device according to any one of clauses 1 to 8,
in which the information processing device is configured to:
   determined quality of the physiological information, based on the physiological information and a quality reference value for evaluating the quality of the physiological information, and
   in a case where the quality of the physiological information during at least a portion of the first period is equal to or lower than a predetermined level:
      replace the physiological information having the quality equal to or lower than the predetermined level with missing information indicating that the physiological information is missing, and
      generate supplementary physiological information for compensating for the missing physiological information based on the physiological information input to the input interface.

Clause 10. The information processing device according to clause 8 or 9,
in which a display mode corresponding to the supplementary physiological information is different from a display mode corresponding to the physiological information input to the input interface.

Clause 11. An information processing method executed by an information processing device including:
classifying physiological information of a subject input to an input interface of the information processing device into a first physiological information group and a second physiological information group, the first physiological information group including the physiological information during at least a first period, the second physiological information group including the physiological information during a second period different from the first period; and
generating display data including the first physiological information group, the second physiological information group, a first line that is a timeline, and a second line that indicates a magnitude of a value of the physiological information,
in which the display data is generated in a form in which the first physiological information group and the second physiological information group are displayed in a state where the first lines overlap each other and the second line is common.

Clause 12. A non-transitory computer readable storage medium storing a computer program including at least one command which, when executed by a processor of an information processing device, causes the information processing device to:
classify physiological information of a subject input to an input interface of the information processing device into a first physiological information group and a second physiological information group, the first physiological information group including the physiological information during at least a first period, the second physiological information group including the physiological information during a second period different from the first period; and
generate display data including the first physiological information group, the second physiological information group, a first line that is a timeline, and a second line that indicates a magnitude of a value of the physiological information,
in which the display data is generated in a form in which the first physiological information group and the second physiological information group are displayed in a state where the first lines overlap each other and the second line is common.

What is claimed is:

1. An information processing device comprising:
an input interface to which physiological information on a subject is input;
at least one processor; and
a memory configured to store at least one command that the processor is configured to execute,
wherein in a case where the at least one command is executed by the processor, the information processing device is configured to:
classifies the physiological information input to the input interface into a first physiological information group and a second physiological information group, the first physiological information group including the physiological information during at least a first period, the second physiological information group including the physiological information during a second period different from the first period; and
generates display data including the first physiological information group, the second physiological information group, a first line that is a timeline, and a second line that indicates a value related to the physiological information; and
the display data is generated in a form in which the first physiological information group and the second physiological information group are displayed in a state where the first lines overlap each other and the second line is common.

2. The information processing device according to claim 1,
wherein a display mode of an overlapped portion which is a portion where a first portion and a second portion overlap is different from a display mode of a non-overlapped portion which is a portion where the first portion and the second portion do not overlap, the first portion being surrounded by the first line and a first graph object indicating a size of each piece of the physiological information included in the first physiological information group, the second portion being surrounded by the first line and a second graph object indicating a size of each piece of the physiological information included in the second physiological information group.

3. The information processing device according to claim 1,
wherein the first period and the second period have the same length,
the information processing device is configured to calculate a comparison value between a first value based on the first physiological information group and a second value based on the second physiological information group, and
in a case where the comparison value is equal to or greater than a threshold value, the information processing device is configured to generate alert information for prompting confirmation of a condition of the subject.

4. The information processing device according to claim 2,
wherein the non-overlapped portion in the first portion is colored with a first color,
the non-overlapped portion in the second portion is colored with a second color different from the first color, and
the overlapped portion is colored with a color different from the first color and the second color.

5. The information processing device according to claim 3,
wherein the first period is a period temporally later than the second period, and
the information processing device is configured to set the threshold value, based on a change tendency of the first physiological information group.

6. The information processing device according to claim 3,
wherein the information processing device is configured to set the threshold value, based on at least one of previous history information, attribute information, condition information, or administration information of the subject.

7. The information processing device according to claim 1,
wherein the information processing device is configured to:
calculate a change value of the previous physiological information, and
in a case where the change value is equal to or greater than a predetermined value, calculate sudden change information indicating that a condition of the subject has suddenly changed.

8. The information processing device according to claim 1,
wherein in a case where the first physiological information group includes missing information indicating that the physiological information is missing during at least a portion of the first period, the information processing device is configured to generate supplementary physiological information for compensating for the missing physiological information based on the physiological information input to the input interface.

9. The information processing device according to claim 1,
wherein the information processing device is configured to:
determined quality of the physiological information, based on the physiological information and a quality reference value for evaluating the quality of the physiological information, and
in a case where the quality of the physiological information during at least a portion of the first period is equal to or lower than a predetermined level:
replace the physiological information having the quality equal to or lower than the predetermined level with missing information indicating that the physiological information is missing, and
generate supplementary physiological information for compensating for the missing physiological information based on the physiological information input to the input interface.

10. The information processing device according to claim 8,
wherein a display mode corresponding to the supplementary physiological information is different from a display mode corresponding to the physiological information input to the input interface.

11. An information processing method executed by an information processing device comprising:
classifying physiological information of a subject input to an input interface of the information processing device into a first physiological information group and a second physiological information group, the first physiological information group including the physiological information during at least a first period, the second physiological information group including the physiological information during a second period different from the first period; and
generating display data including the first physiological information group, the second physiological information group, a first line that is a timeline, and a second line that indicates a magnitude of a value of the physiological information,
wherein the display data is generated in a form in which the first physiological information group and the second physiological information group are displayed in a state where the first lines overlap each other and the second line is common.

12. A non-transitory computer readable storage medium storing a computer program comprising at least one command which, when executed by a processor of an information processing device, causes the information processing device to:
classify physiological information of a subject input to an input interface of the information processing device into a first physiological information group and a second physiological information group, the first physiological information group including the physiological information during at least a first period, the second physiological information group including the physiological information during a second period different from the first period; and
generate display data including the first physiological information group, the second physiological information group, a first line that is a timeline, and a second line that indicates a magnitude of a value of the physiological information,
wherein the display data is generated in a form in which the first physiological information group and the second physiological information group are displayed in a state where the first lines overlap each other and the second line is common.
